(19) United States
(12) Patent Application Publication
(10) Pub. No.: US 2007/0196528 A1
(43) Pub. Date: Aug. 23, 2007

(54) Cosmetic composition based on caper flower bud extract

(75) Inventors: Estelle Bonnet, Villefontaine (FR); Alain Guichon, Lyon (FR); Virginie Charton, Villeurbanne (FR); Vincent Hubiche, Lyon (FR); Paula Lennon, Lyon (FR)

Correspondence Address: HESLIN ROTHENBERG FARLEY & MESITI PC, 5 COLUMBIA CIRCLE, ALBANY, NY, 12203, US

(73) Assignee: GATTEFOSSE S.A.S., Saint Priest (FR)

(21) Appl. No.: 10/599,909

(22) PCT Filed: Mar. 21, 2005
(86) PCT No.: PCT/FR05/50180

§ 371 Date: Oct. 13, 2006

(30) Foreign Application Priority Data
Apr. 16, 2004 (FR) 04.04152

(51) Int. Cl. A61K 36/31

(52) U.S. Cl. 424/778

(57) ABSTRACT

A caper flower bud extract is obtained by supercritical fluid extraction. The supercritical fluid preferably contains carbon dioxide and at least one cosolvent. A cosmetic composition contains between 0.1 and 10% of the extract.

The invention concerns a caper flower bud extract. It also deals with a cosmetic composition containing said extract.

The document “Isolation and identification of an anti-inflammatory principle from Capparis spinosa”, Department of Pharmacognosy, College of Pharmacy, King Saud University, Riyadh, Saudi Arabia, Pharmazie 43 (1988), H.9, sheds light on the anti-inflammatory properties of caper leaves. This property is demonstrated on oedemas in rats using an alcohol extract of dried Capparis spinosa leaves. The molecules identified as providing this anti-inflammatory activity are designated as Cappaprenol-12, Cappaprenol-13 and Cappaprenol-14.

The document “Anti-inflammatory activity of some Saudi Arabian medicinal plants”, College of Pharmacy, King Saud University, Riyadh, Saudi Arabia, Agents and Actions, vol. 17, 3/4 (1985) proposes, for treating rheumatism or arthritis, the use of an ethanolic extract of the caper bush, notably Capparis decidua or an aqueous extract obtained from Capparis spinosa. In this document, the aerial parts of the plant are used.

Unlike alcoholic extracts, aqueous extracts do not contain cappaprenol and are therefore less active. Alcoholic extracts, however, have the disadvantage of containing a large amount of undesirable molecules (glucosinolates) which, after enzymatic hydrolysis, give D-glucose-type molecules, sulphate ions, and sulphur and/or nitrolised compounds such as thiocyanates, isothiocyanates or oxazolidinethiones, giving the extract a pungent odour. In other terms, the extracts proposed in these documents are incompatible with cosmetic applications.

The LEMMI CENA document “Ricerche sperimentali sull'azione cosmetologica dei Capperi”, Vol. 61, no. 1, 1979, pages 2-9, describes an extract of dehydrated capers obtained by vacuum extraction with acetone; the substance obtained is then hydrated with water at 80° C. According to this process, all of the active molecules are retrieved and notably bioflavonoids such as rutin, as well as molecules such as glucosides, whose degradation products (notably isocyanates) are undesirable.

The problem that the invention claims to solve is to develop extracts which do not have these disadvantages.

The Applicant observed that extracts of floral buds corresponding to the actual capers, when they were obtained by supercritical fluid extraction, presented higher cappaprenol concentrations and lower glucosinate concentrations than extracts obtained by maceration or high-vacuum extraction. The extracts thus obtained can be used in cosmetics. Cosmetic compositions containing this extract, applied topically, have demonstrated particularly useful soothing and moisturising properties.

Consequently, the invention concerns a caper flower bud extract which may be obtained by supercritical fluid extraction.

In a preferred embodiment, the flower buds are of the species Capparis spinosa.

The extract obtained notably contains cappaprenol-12, cappaprenol-13 and cappaprenol-14, but also fatty acids such as palmitic acid, oleic acid, linoleic acid and linolenic acid.

The supercritical fluid extraction applied in this invention is described in detail in the document EP-A-1 222 008, included in reference. In practice, the supercritical fluid contains CO₂ and at least one cosolvent. The cosolvent may be any of the extraction solvents used in cosmetics. These may notably include alcohol, certain natural or synthetic oils and fats as listed below. It appears, however, that the use as a cosolvent for fat instead of alcohol gives better results in terms of the quantity of cappaprenol and natural fatty acids extracted and the stability of said extract.

The Applicant observed that quite surprisingly, the use of 2-octyldodecyl myristate (ODM) as a cosolvent was particularly advantageous.

In practice, the cosolvent/caper ratio is between 0.5 and 10, preferably 1.75.

In a preferred embodiment, before the actual extraction, the fresh flower buds are dried or lyophilised. When dried, drying is advantageously performed using hot air, at atmospheric pressure, at a temperature between 25 and 90° C., preferably 60° C., so as to obtain a residual humidity rate of less than 10%.

The dried or lyophilised capers are then ground into a powder using mechanical grinding, advantageously cryogrinding. The ground capers may then be rehydrated with a suitable quantity of water before the extraction step. In practice, the residual humidity rate of the rehydrated capers is between 10 and 40%, advantageously 20%.

The extraction itself is performed at a temperature between 30 and 90° C., advantageously 50° C., at a pressure between 73 and 350 bars, advantageously 290 bars, for 0.5 to 10 hours, advantageously 5 hours.

The crude extract thus obtained is then treated by decantation to eliminate residual water and then filtered, in practice on a cellulose plate.

The invention also concerns as cosmetic composition containing the caper flower bud extract described above.

In practice, the extract accounts for between 0.1 and 10% by weight of the composition, advantageously between 0.3 and 3% by weight.

The cosmetic composition of the invention is generally applied topically and is used for its soothing and moisturising properties. It also has highly appreciated qualities of penetration, suppleness and mildness.

The composition according to the invention may come in all of the pharmaceutical forms normally used for a topical application on the skin or hair, notably in the form of an aqueous solution, an oil-in-water or water-in-oil or multiple emulsion, a silicone emulsion, a microemulsion or nanoemulsion, or an aqueous gel.

This composition may be more or less fluid and have an appearance that is, amongst others, a white or coloured cream, an ointment, a milk, a lotion, a serum or a gel.

The composition of the invention may contain the usual adjuvants used in the cosmetic and dermatological fields, such as fats, emulsifiers and co-emulsifiers, hydrophilic or lipophilic gelling agents, hydrophilic or lipophilic active ingredients, preservatives, antioxidants, solvents, fragrances, fillers, hydrophilic and lipophilic filters, dyestuffs, neutralising agents, propenetrating agents and polymers.

The quantities of these various adjuvants are those conventionally used in the fields in question and, for example, between 0.01 and 30% of the total weight of the composition. These adjuvants, depending on their nature, may be added in the oil phase or in the aqueous phase.

The fats that can be used in the invention include mineral oils, oils of animal origin (lanolin), vegetable oils, synthetic oils (isopropyl myristate, octyldodecyl, isostearyl isostearate, decyl oleate or isopropyl palmitate), silicone oils (cyclomethicone, dimethicone) and fluorinated oils. The following can be used as fats: fatty alcohols, fatty acids, waxes and gums and notably silicone gums and elastomers.

As emulsifiers and co-emulsifiers that can be used in the invention, we can mention, for example, polyglycerol fatty acid esters, sucrose fatty acid esters, sorbitan fatty acid esters, oxyethylenated sorbitan fatty acid esters, PEG fatty acid ethers, glycerol fatty acid esters, alkyl sulphates, alkyl ether sulphates, alkyl phosphates, alkyl polyglucosides and dimethicone copolyols.

As hydrophilic gelling agents, we can notably mention carboxyvinyl polymers (carbomer), acrylic copolymers such as acrylate/alkylacrylate copolymers, polyacrylamides, polysaccharides such as xanthan gum, guar gum, natural gums such as cellulose gum and derivatives, clays and 2-acrylamido-2-methylpropane acid copolymers.

As lipophilic gelling agents we can mention modified clays such as bentones, fatty acid metal salts, hydrophobic silica and ethylcellulose.

Cosmetic composition may also contain other active ingredients. As active ingredients we can notably use depigmenting agents, emollients, moisturisers, antiseborrheic agents, antiacne agents, keratolytic and/or desquamative agents, anti-wrinkle agents and tensors, draining agents, anti-irritation agents, soothing agents, slimming products such as xanthic bases (caffeine), vitamins and their combinations, matting agents, anti-ageing ingredients such as retinol, anti-wrinkle agents, and essential oils.

In case of incompatibility between them or with the caper flower bud extract, the aforementioned active ingredients and/or flower bud abstract may be added in spheroids, notably ionic or non-ionic capsules and/or micro- or nanoparticles (micro/nanocapsules and/or micro/nanospheres), so as to isolate them from each other in the composition.

Among the preservatives that can be used according to the invention, we can mention benzoic acid, its salts and its esters; sorbic acid and its salts; parabens, their salts and esters; triclosan; imidazolidinyl urea; phenoxyethanol; DMDM hydantoin; diazolidinyl urea; chlorphenesin.

Antioxidants that can be used according to the invention include chelating agents such as EDTA and its salts.

Solvents that can be used according to the invention include water, ethanol, glycerine, propylene glycol, butylene glycol and sorbitol.

Fillers that can be used according to the invention include talc, kaolin, mica, serecite, magnesium carbonate, aluminium silicate, magnesium silicate and organic powders such as nylon.

Filters that can be used according to the invention include conventionally used UVA and UVB filters such as benzophenone-3, butyl methoxydibenzoyl methane, octocrylene, octyl methoxycinnamate, 4-methylbenzylidene camphor, octyl salicylate, tacephthalydene dicamphor sulphanic acid and drometrizole trisiloxane. We can also mention the physical filters TiO2 and ZnO in their micrometric and nanometric forms.

Dyestuffs that can be used according to the invention include lipophilic dyes, hydrophilic dyes, pigments and mother-of-pearl commonly used in cosmetic or dermatological compositions, and their combinations.

Neutralising agents that can be used according to the invention include soda, triethanolamine, aminomethyl propanol and potassium hydroxide.

Propenetrating agents that can be used according to the invention include alcohols and glycols (ethanol, propylene glycol), ethoxydiglycol, fatty acid alcohols (oleic acid), fatty acid esters, dimethyl isosorbide.

The composition according to the invention may be used as a care product, a cleaning product and/or a skin make-up product, as a sun protection product or as a hair product, for example as a shampoo or conditioner.

The invention and the advantages it provides will become clearer with the following examples of its embodiment.

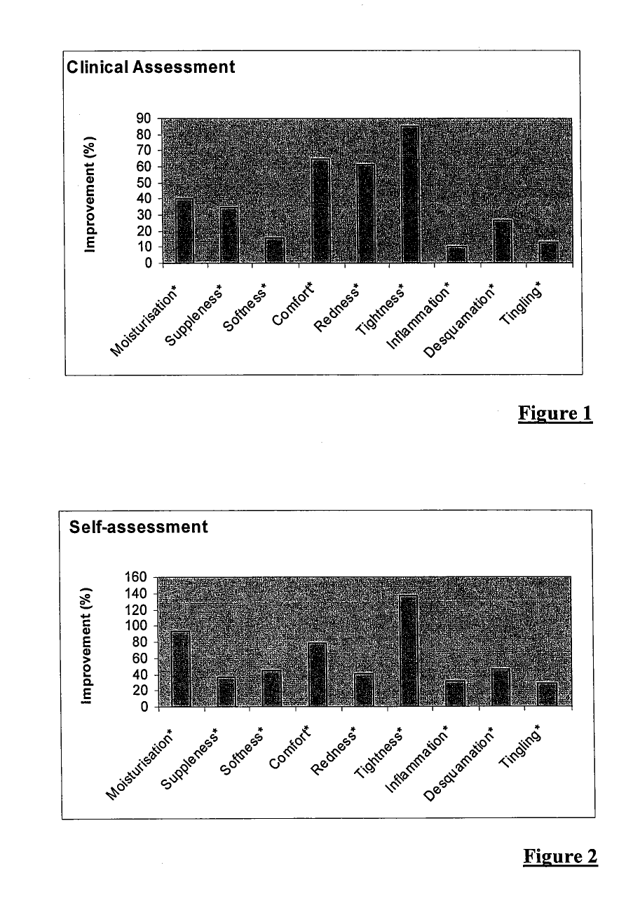

FIGS. 1 and 2 represent the mean percentages of improvement of the effectiveness criteria assessed clinically by a dermatologist (FIG. 1) or by self-assessment (FIG. 2).

### EXAMPLE 1

### Extraction Procedure

The caper extract is obtained from caper flower buds of the species Capparis spinosa.

Before the extraction step, the fresh capers are dried in a hot air current, at a temperature of approximately 60° C., so as to obtain a residual humidity rate in the capers of less than 10%. The dried capers are then ground into a powder by cryogrinding. The ground capers are then rehydrated with a suitable amount of water before the extraction step. Moistening the plant provides better extraction power with supercritical CO₂. The residual humidity rate of the rehydrated capers is approximately 20%.

The actual extraction is performed by a supercritical fluid comprising CO₂ in the presence of 2-octyldodecyl myristate (ODM) as the cosolvent.

The operating conditions are as follows:
ODM/caper ratio: 1.75
temperature: 50° C.
pressure: 290 bars
time: 5 hrs

The crude extract is then treated by decantation to eliminate the residual water and then filtered on cellulose plates to 5 μm.

### EXAMPLE 2

### Comparison of the Extractive Power of ODM Depending on the Extraction Technique

| | ODM | ODM | ODM |
|---|---|---|---|
| | supercritical CO₂ | microwaves | normal heating |
| Cappaprenols | 42.0 | 9.0 | 6.4 |
| (mg/100 g extract) | | | |

Comment: the extraction temperature is identical for the 3 techniques (50° C.).

The supercritical CO₂ extraction technique provides a cape extract that is 4.7 times more concentrated that the extract obtained with microwaves and 6.6 times more concentrated than the extract obtained by conventional hot maceration.

### EXAMPLE 3

### Comparison of the Extractive Power of ODM and Ethanol used with Supercritical CO₂

| | Ethanol | ODM |
|---|---|---|
| Cappaprenols | 15.0 | 42.0 |
| (mg/100 g extract) | | |
| Cappaprenols | 10.8 | 32.7 |
| (mg/100 g capers, dry weight) | | |

For identical operating conditions, excluding the nature of the cosolvent, supercritical CO₂ extraction with ODM as the cosolvent extracts 3 times as much cappaprenols as the same extraction using supercritical CO₂ with ethanol as the cosolvent.

### EXAMPLE 4

### Stability of an Extract obtained in ODM or Ethanol used with Supercritical CO₂

Stability was monitored on samples in which no preservatives and/or additives were used.

| | | Ethanol | ODM |
|---|---|---|---|
| | | Cappaprenol | Cappaprenol |
| | | content | content |
| | Storage | (mg/100 g | (mg/100 g |
| Analysis date | temperature | extract) | extract) |
| D₀ (Day of | Ambient | 3.7 | 21 |
| extraction) | temperature | | |
| | (A.T.) | | |
| D₀ + 30 days | 4° C. | 3 | 17.8 |
| | A.T. | 3 | 21 |
| | 40° C. | 2.8 | 15.3 |
| D₀ + 60 days | 4° C. | 0.7 | 17.6 |
| | A.T. | 0.6 | 19 |
| | 40° C. | traces | 14.8 |

For identical storage conditions, ODM is a medium in which cappaprenols are much more stables than in ethanol.

### EXAMPLE 5

### Solubility of Glucosinates in Water, Alcohol and ODM

| | ODM | water | alcohol |
|---|---|---|---|
| Glucosinate | insoluble | soluble | slightly soluble |

The insolubility of glucosinates in ODM provides extracts containing no substances from the hydrolysis of this molecule, which have a pungent odour and are therefore unsuitable for cosmetics.

### EXAMPLE 6

### Efficacy Test on a Cream Based on the Extract in the Invention Applied in vivo on a Panel of 20 Volunteers with Sensitive Skin

The aim is to assess the efficacy and the cosmetic acceptability, under normal conditions of use, of a soothing protective cream containing 2% extract Capparis spinosa flower buds.

There were 20 panelists who had sensitive, reactive skin, subject to sensations of discomfort and diffuse redness. The substance was applied twice daily for 28 days.

Three series of results are presented:
Clinical assessment by the dermatologist at T0 then at T4 weeks of the effectiveness criteria claimed on a 10-point analogous scale running from 0 to 9 (the better the assessment of the parameter, the closer the mark is to 9).
Self-assessment at T0 and at T4 weeks by the volunteer following the same protocol as before.
The subjects answered an acceptability questionnaire at T4 weeks.

Statistical analysis of the results is performed and significant data are identified: (*) significant improvement p<5 %.

| Question | Percentage of satisfaction |
|---|---|
| The texture is comfortable | 70% |
| The product penetrates quickly | 75% |
| My skin is better moisturised | 85% |
| My skin is more comfortable | 60% |
| My skin is soothed | 75% |
| My skin is supple | 80% |
| My skin is soft | 95% |
| Redness is attenuated | 65% |
| My skin is protected better | 75% |
| My skin is less reactive | 75% |
| Sensations of discomfort are attenuated | 75% |
| Sensations of tightness are attenuated | 90% |

FIGS. 1 and 2 represent the mean percentages of improvement of the effectiveness criteria assessed clinically by a dermatologist (FIG. 1) or by self-assessment (FIG. 2).

### EXAMPLE 7

### Formulations

Example of Formulation 1

Soothing slimming body milk

| Composition | Quantity (%) |
|---|---|
| PEG-6 Stearate, Ceteth-20, Steareth-20 | 8.0 |
| Propylene Glycol Dipelargonate | 10.0 |
| Stearic acid | 1.0 |
| Hydrogenated castor oil | 1.0 |
| Kernel oil | 3.0 |
| Dimethicone | 2.0 |
| Tocopherol Acetate | 0.5 |
| Polydecene | 3.0 |
| Cyclomethicone | 3.0 |
| Preservative | 1.0 |
| Carbomer | 0.15 |
| Xanthan gum | 0.3 |
| Alcohol | 5.0 |
| Glycerine | 3.0 |
| Sodium hydroxide (10% solution) | 0.3 |
| Extract of Microtea debilis | 3.0 |
| Extract of Capparis spinosa flower buds | 1.5 |
| Ascorbic Acid | 0.05 |
| Fragrance | 0.4 |
| Water | to 100 |

Example of formulation 2

Anti-age cream

| Composition | Quantity (%) |
|---|---|
| Preservative | 1 |
| Carbomer | 0.4 |
| Glycerine | 3 |
| Xanthan gum | 0.1 |
| Polysorbate-60 | 0.9 |
| Glyceryl Stearate, PEG-100 Stearate | 2.1 |
| Cetyl Alcohol | 2.6 |
| Paraffin Oil | 7.5 |
| Isopropyl Myristate | 7.5 |
| Ethoxydiglycol | 5 |
| Extract of Capparis spinosa flower buds | 1 |
| Retinyl Palmitate | 0.5 |
| Fragrance | 0.2 |
| Water | to 100 |
| Triethanolamine | 0.3 |

Example of Formulation 3

Rich W/O cream

| Composition | Quantity (%) |
|---|---|
| Glycerine | 3 |
| Preservative | 1 |
| Magnesium Sulphate | 0.7 |
| Cetyl Dimethicone Copolyol | 2.5 |
| Isohexadecane | 5 |
| Caprylic/Capric Triglyceride | 5 |
| Dimethicone | 5 |
| Alcohol | 5 |
| Extract of Capparis spinosa flower buds | 2 |
| Fragrance | 0.1 |
| Water | to 100 |

Example of Formulation 4

Microemulsion for sensitive skin

| Composition | Quantity (%) |
|---|---|
| PEG-8 Caprylic/Capric Glycerides | 13.33 |
| Polyglyceryl-6 Dioleate | 8.67 |
| Isostearyl Isostearate | 4 |
| Cyclomethicone | 2.3 |
| Diisopropyl Adipate | 1.6 |
| Octyldodecanol | 2 |
| PPG-5 Ceteth-20 | 2 |
| Preservative | 0.4 |
| Ethoxydiglycol | 2 |
| Extract of Capparis spinosa flower buds | 1 |
| Water | to 100 |

Example of Formulation 5

Multiple W/O/W emulsion

| Composition | Quantity (%) |
|---|---|
| PEG-30 Dipolyhydroxystearate | 2.4 |
| Isohexadecane | 9 |
| PPG-15 Stearyl Ether | 4.5 |
| Caprylic/Capric Triglyceride | 4.5 |
| Magnesium Sulphate | 0.82 |
| Preservative | 1.2 |
| Extract of Capparis spinosa flower buds | 2 |
| Poloxamer 407 | 2 |
| Glycerine | 3 |
| Xanthan gum | 0.7 |
| Fragrance | 0.2 |
| Water | to 100 |

Example of Formulation 6

Spray sunscreen with chemical filters

| Composition | Quantity (%) |
|---|---|
| Glyceryl Stearate, Ceteareth-20, Ceteareth-12, | 5 |
| Cetearyl Alcohol, Cetyl Palmitate | |
| Ceteareth-20 | 2 |
| Caprylic/Capric Triglyceride | 5 |
| Squalane | 3 |
| C12/C15 Alkyl Benzoate | 4 |
| Octyl Methoxycinnamate | 5 |
| Butyl Methoxydibenzoylmethane | 2 |
| Preservative | 1 |
| Water | to 100 |
| Glycerine | 5 |
| Bisabolol | 0.3 |
| Extract of Capparis spinosa flower buds | 1 |

Example of Formulation 7

High SPF sunscreen

| Composition | Quantity (%) |
|---|---|
| DEA Cetyl Phosphate | 2 |
| Glyceryl Stearate, PEG-100 Stearate | 4 |
| Beeswax | 2 |
| Octyl Methoxycinnamate | 7 |
| Butyl Methoxydibenzoylmethane | 2 |
| Polysilicone-15, Methyl Alcohol, Alcohol | 2 |
| Benzophenone-3 | 1 |
| Titanium Dioxide | 3 |
| C12/C15 Alkyl Benzoate | 3 |
| Cyclomethicone | 2 |
| Tocopherol Acetate | 0.5 |
| EDTA | 0.1 |
| Acrylates/C10-30 Alkyl Acrylate Crosspolymer | 0.2 |
| Xanthan gum | 0.3 |
| Preservative | 1 |
| Butylene Glycol | 3 |
| Extract of Capparis spinosa flower buds | 1 |
| Sodium hydroxide (10% solution) | 0.4 |
| Fragrance | 0.3 |
| Water | to 100 |

Example of Formulation 8

Children's sunscreen

| Composition | Quantity (%) |
|---|---|
| Tribehenin PEG-20 Esters | 2.5 |
| PEG-6 Stearate, Ceteth-20, Steareth-20 | 3.5 |
| Octyldodecyl Myristate | 4 |
| Cyclomethicone | 4 |
| Preservative | 0.7 |
| Glycerine | 3 |
| Water | to 100 |
| Xanthan gum, Hectorite, Cellulose | 0.5 |
| Zinc Oxide | 3 |
| Titanium Dioxide | 5 |
| Extract of Capparis spinosa flower buds | 1 |

Example of Formulation 9

Make-up foundation

| Composition | Quantity (%) |
|---|---|
| Glyceryl Stearate, Propylene Glycol Stearate, | 5 |
| Glyceryl Isostearate, Propylene Glycol Isostearate, | |
| Oleth-25, Ceteth-25 | |
| Glyceryl Dibehenate, Tribehenin, | 1 |
| Glyceryl Behenate | |
| Ethoxydiglycol Oleate | 7.5 |
| Isostearyl Isostearate | 5 |
| Cetostearyl Alcohol | 2 |
| Dimethicone | 5 |
| Tocopherol Acetate | 0.5 |
| Preservative | 0.6 |
| Xanthan gum | 0.4 |
| Microcrystalline Cellulose, Cellulose Gum | 1.5 |
| Titanium Dioxide | 6.6 |
| Iron Oxides (Yellow pigment) | 1.55 |
| Iron Oxides (Red Pigment) | 0.43 |
| Iron Oxides (Black pigment) | 0.11 |
| Ethoxydiglycol Oleate | 2.5 |
| Dimethicone, Dimethiconol | 3 |
| Alcohol | 5 |
| Extract of Capparis spinosa flower buds | 2 |
| Water | to 100 |

Example of Formulation 10

Shampoo

| Composition | Quantity (%) |
|---|---|
| Acrylates Copolymer | 1.5 |
| Sodium Lauryl Sulphate | 5 |
| Sodium Laureth Sulphate | 4 |
| Cocamidopropyl Betaine | 1.5 |
| Polyquaternium-10 | 0.25 |
| DMDM Hydantoin | 0.3 |
| Sodium hydroxide (20% solution) | 1.3 |
| Citric Acid (50% solution) | 0.7 |
| Extract of Capparis spinosa flower buds | 0.5 |
| Fragrance | 0.5 |
| Sodium Chloride | 0.5 |
| Water | to 100 |

Example of Formulation 11

Soothing stick

| Composition | Quantity (%) |
|---|---|
| Isostearyl Isostearate, Ozokerite, Castor | 96.2 |
| (Ricinus communis) Oil, Hydrogenated Palm Kernel | |
| Glycerides, Propylene Glycol Dipelargonate, | |
| Polyglyceryl-2 Sesquiisostearate, Polyglyceryl-2 | |
| Sesquistearate, PEG-8 Beeswax, Phenyl Trimethicone, | |
| Propylene Glycol Isostearate, Cetyl Lactate, | |
| Hydrogenated Palm Glycerides | |
| Mineral Oil, Vegetable Oil, Aloe | 1 |
| Extract | |
| Extract of Capparis spinosa flower buds | 1 |
| Mica, Titanium Dioxide | 1.5 |
| Fragrance | 0.3 |

Example of Formulation 12

Soothing protective cream

| Composition | Quantity (%) |
|---|---|
| Cetyl Alcohol, Glyceryl Stearate, PEG-75 Stearate, | 6 |
| Ceteth-20, Steareth-20 | |
| Octyldodecyl Myristate | 5 |
| Cyclomethicone | 4 |
| Preservative | 0.7 |
| Water | to 100 |
| Carbomer | 0.15 |
| Xanthan gum | 0.3 |
| Glycerine | 3 |
| Aminomethyl Propanol | 0.15 |
| Aluminium Starch Octenyl Succinate | 4 |
| Extract of Capparis spinosa flower buds | 2 |

Example of Formulation 13

Eye and face make-up remover

| Composition | Quantity (%) |
|---|---|
| Water | to 100 |
| Acrylate/C10-30 Alkyl Acrylate Crosspolymer | 0.25 |
| Xanthan gum | 0.2 |
| Sodium hydroxide | 0.5 |
| Water, Glycerin, Oat (Avena sativa) Extract | 2 |
| Preservative | 0.3 |
| Isopropyl Palmitate | 2 |
| Mineral Oil | 5 |
| Cyclomethicone | 2.5 |
| Caprylic/Capric Triglyceride | 3.5 |
| Isostearyl Alcohol, Butylene Glycol Cocoate, | 4 |
| Ethylcellulose | |
| Extract of Capparis spinosa flower buds | 1 |

Example of Formulation 14

Protective toner

| Composition | Quantity (%) |
|---|---|
| Water | to 100 |
| Butylene Glycol | 3 |
| Glycerine | 3 |
| Preservative | 0.3 |
| Sodium PCA | 1 |
| Smithsonite Extract | 2.5 |
| CI 42090 | 0.03 |
| CI 19140 | 0.03 |
| Fragrance | 0.2 |
| Extract of Capparis spinosa flower buds | 0.5 |
| PEG-40 Hydrogenated Castor Oil, Polysorbate 20, | 1.2 |
| Octoxynol-11 | |

Example of Formulation 15

Foaming gel

| Composition | Quantity (%) |
|---|---|
| Water | to 100 |
| Acrylate/C10-30 Alkyl Acrylate Crosspolymer | 0.7 |
| Sodium hydroxide | 0.2 |
| PEG-2 Stearate | 1.5 |
| Sodium Laureth Sulphate | 10 |
| PEG-150 Distearate | 0.5 |
| Ethoxydiglycol Oleate | 3 |
| Cocamidopropyl Betaine | 3 |
| Decyl Glucoside | 4 |
| Styrene/PVP Copolymer | 1 |
| Sodium hydroxide | 1.2 |
| Fragrance | 0.5 |
| Preservative | 0.5 |
| Extract of Capparis spinosa flower buds | 0.5 |

Example of Formulation 16

Anti-ageing cream

| Composition | Quantity (%) |
|---|---|
| Water | to 100 |
| Carbomer | 0.25 |
| Xanthan gum | 0.2 |
| Dimethicone | 4 |
| Octyl Methoxycinnamate | 5 |
| Benzophenone-3 | 1 |
| Ethoxydiglycol Oleate | 6 |
| Tocopheryl Acetate | 0.5 |
| Cetyl Alcohol | 2.5 |
| Stearyl Alcohol | 2.5 |
| Propylene Glycol, Ethylcellulose, Propylene Glycol | 6 |
| Isostearate | |
| Aluminium Starch Octenyl Succinate | 4 |
| Glycerine | 2 |
| Butylene Glycol | 2 |
| Dimethicone, Dimethiconol | 2 |
| Extract of Fagus salvatica | 2 |
| Extract of Capparis spinosa flower buds | 1 |
| CI 15985 | 0.4 |
| Preservative | 0.4 |
| Fragrance | 0.15 |
| Aminomethyl Propanol | 0.28 |

Example of Formulation 17

Soothing cream

| Composition | Quantity (%) |
|---|---|
| Glyceryl Stearate, Propylene Glycol Stearate, | 5 |
| Glyceryl Isostearate, Propylene Glycol Isostearate, | |
| Oleth-25, Ceteth-25 | |
| Glyceryl Dibehenate, Tribehenin, Glyceryl Behenate | 1 |
| Cyclomethicone | 5 |
| Myreth-3 Myristate | 2 |
| Phenoxyethanol, Methylparaben, Butylparaben, | 0.5 |
| Ethylparaben, Propylparaben | |
| C10-30 Cholesterol/Lanosterol Esters | 2 |
| Cetostearyl Alcohol | 3 |
| Isopropyl Jojobate, Jojoba Alcohol | 3 |
| Stearic Acid | 1.5 |
| Hydrogenated Colza Seed Oil | 2 |
| Actinidia chinensis (Kiwi) Fruit Water | to 100 |
| Cetearyl Octanoate | 5 |
| Tocopherol Acetate | 1 |
| Glycerine | 8 |
| Acrylates Copolymer | 0.9 |
| Polyacrylamide, Isoparaffin, Laureth-7 | 0.7 |
| Sodium hydroxide | 0.25 |
| Pilewort Extract | 0.2 |
| Extract of Capparis spinosa flower buds | 3 |
| Fragrance | 0.2 |

Example of Formulation 18

Babycare cream

| Composition | Quantity (%) |
|---|---|
| Polyglyceryl-3 Diisostearate | 5 |
| Mineral Oil, Apricot (Primus armeniaca) Kernel Oil, | 2 |
| Matricaria (Chamomilla recutita) Extract | |
| Sweet almond oil | 2 |
| Mineral oil | 15 |
| Extract of Capparis spinosa flower buds | 1 |
| Zinc Oxide | 10 |
| Caprylic/Capric Triglycerides, Tocopherol, Propyl | 0.04 |
| Gallate, Citric Acid | |
| Phenoxyethanol, Methylparaben, Butylparaben, | 0.4 |
| Ethylparaben, Propylparaben | |
| Water | to 100 |
| Glycerine | 5 |
| Sodium Chloride | 0.5 |
| Magnesium Sulphate | 0.5 |
| Allantoin | 0.1 |

Example of Formulation 19

Skincare oil

| Composition | Quantity (%) |
|---|---|
| Caprylic/Capric Triglyceride | 40 |
| Propylene Glycol Dipelargonate | 25 |
| Sweet avocado oil | 2 |
| Mineral oil | 20 |
| Vitamin E acetate | 0.5 |
| Extract of Capparis spinosa flower buds | 1.5 |
| Fragrance | 1 |

Example of Formulation 20

Slimming oil

| Composition | Quantity (%) |
|---|---|
| Caprylic/Capric Triglyceride | 30 |
| Octyl dodecyl myristate | 20 |
| Propylene Glycol Dipelargonate | 30 |
| Cyclomethicone | 16.95 |
| Extract of Microtea debilis | 0.05 |
| Extract of Capparis spinosa flower buds | 2 |
| Fragrance | 1 |

Example of Formulation 21

Solid make-up foundation

| Composition | Quantity (%) |
|---|---|
| Polyglyceryl-3 Diisostearate | 2.5 |
| Hydrogenated castor oil | 0.5 |
| Polydecene | 7 |
| Preservative | 1 |
| Ethoxydiglycol Oleate | 4.5 |
| Titanium dioxide | 7.5 |
| Iron oxides | 2.6 |
| Water | to 100 |
| Sodium chloride | 0.25 |
| Magnesium sulphate | 0.25 |
| Ethoxydiglycol Behenate | 7 |
| Glyceryl Dibehenate + Tribehenin + Glyceryl | 8 |
| Behenate | |
| Cyclomethicone | 21 |
| Starch powder | 12 |
| Extract of Capparis spinosa flower buds | 4 |
| Fragrance | 0.2 |

Example of Formulation 22

Make-up remover oil

| | |
|---|---|
| Isopropyl palmitate | 30 |
| Propylene Glycol Dipelargonate | 30 |
| Cyclomethicone | 15 |
| POE oleyl alcohol ether | 10 |
| Extract of Capparis spinosa flower buds | 5 |

Example of Formulation 23

Shaving cream

| | |
|---|---|
| Stearic acid | 4.5 |
| Coconut oil fatty acid | 1.5 |
| Glycerol monostearate | 5 |
| Glycerine | 10 |
| Triethanolamine | 4 |
| Extract of Capparis spinosa flower buds | 0.5 |
| Water | to 100 |
| Propulsion gas: nitrogen | |

Example of Formulation 24

Sugar gumming

| | |
|---|---|
| PEG-8 Beeswax | 8.5 |
| Cetyl alcohol + Ceteth-20 + Steareth-20 | 2 |
| Glyceryl Dibehenate + Tribehenin + Glyceryl behenate | 2.5 |
| Hydrogenated castor oil | 1 |
| Cetyl dimethicone | 2.5 |
| Cyclomethicone | 4 |
| Caprylic/Capric Triglyceride PEG-4 esters | 6 |
| Propylene Glycol Dipelargonate | 4 |
| Ethoxydiglycol Oleate | 3 |
| Vitamin E acetate | 0.5 |
| Octyl dodecyl myristate | 3 |
| Preservative | 1 |
| Glycerin | 14.5 |
| Butylene Glycol | 13 |
| Water | to 100 |
| NaCl | 6.5 |
| Polyacrylamide + Isoparaffin + Laureth-7 | 2.5 |
| Extract of Capparis spinosa flower buds | 1 |
| Perfume | 0.5 |
| Sucre | 22 |

Example of Formulation 25

Soothing anti-hair growth cream

| | |
|---|---|
| Tribehenin PEG-20 Esters | 6 |
| Octyl dodecyl myristate | 5 |
| Squalane | 5 |
| Octyl palmitate | 2 |
| Apricot oil | 2 |
| Isostearyl isostearate | 3 |
| PDMS crosspolymer | 2 |
| Preservative | 1 |
| Water | to 100 |
| Glycerine | 5 |
| Extract of Saw palmetto, Epilobium and Pumpkin | 5 |
| (ARP 100 by Greentech) | |
| Extract of Capparis spinosa flower buds | 3 |

1. A method for obtaining an extract of caper flower buds comprising: providing caper flower buds, and supercritical fluid extraction.

2. The method as claimed in claim 1, wherein the flower buds are of the species Capparis spinosa.

3. The method as claimed in claim 1, wherein the supercritical fluid contains CO₂ and at least one cosolvent.

4. The method as claimed in claim 3, wherein the cosolvent comprises 2-octyldodecyl myristate (ODM).

5. The method as claimed in claim 1, wherein fresh flower buds are first dried with hot air, at atmospheric pressure, at a temperature between 25 and 90° C., and then ground and rehydrated.

6. The method as claimed in claim 1, wherein the extraction is performed at a temperature between 30 and 90° C., at a pressure between 73 and 350 bars, for a period of 0.5 to 10 hours.

7. The method as claimed in claim 6, wherein the extract is then treated by decantation and then filtered.

8. A method for producing a cosmetic composition, comprising: obtaining the extract in accordance with claim 1, and including the extract in a cosmetic composition.

9. The method as claimed in claim 8, wherein the extract accounts for between 0.1 and 10% by weight of the composition.

10. The method as claimed in claim 8, further comprising: including in the composition at least one active ingredient selected from the group consisting of depigmenting agents, emollients, moisturisers, antiseborrheic agents, antiacne agents, keratolytic and desquamative agents, anti-wrinkle agents and tensors, draining agents, anti-irritation agents, soothing agents, slimming products, vitamins and their combinations, matting agents, anti-ageing ingredients, anti-wrinkle agents, and essential oils.

11. The method as claimed in claim 8, further comprising: including in the composition UVA and UVB filters selected from the group consisting of benzophenone-3, butyl methoxydibenzoyl methane, octocrylene, octyl methoxycinnamate, 4-methylbenzylidene camphor, octyl salicylate, tacephthalydene dicamphor sulphanic acid and drometrizole trisiloxane.

12. The method as claimed in claim 5, wherein the temperature is 60° C.

13. The method as claimed in claim 6, wherein the temperature is 50° C., the pressure is 290 bars, and the period is 5 hours.

14. The method as claimed in claim 9, wherein the extract accounts for between 0.3 and 3% by weight of the composition.

15. A composition comprising cappaprenols 12, 13 and 14 and a fatty acid.

16. A composition comprising from 14.8 to 42 mg of at least one of cappaprenols 12, 13 and 14 in 100 g of a fat.

17. A composition according to claim 15 wherein said fatty acid comprises at least one of palmitic acid, oleic acid, linoleic acid and linolenic acid.

18. A glucosinate-free composition according to claim 15.

19. A composition according to claim 15 prepared by the process of extracting flower buds of the species Capparis spinosa with a mixture of supercritical CO₂ and octyldodecyl myristate.

20. A cosmetic composition comprising a composition according to claim 15 and a pharmaceutical carrier for topical application.
